(19) United States
(12) Patent Application Publication
(10) Pub. No.: US 2017/0165048 A1
(43) Pub. Date: Jun. 15, 2017

(54) Device for Changing course of vessel and treatment method using the same

(71) Applicants: Industry-Academic Cooperation Foundation, Yonsei University, Seoul (KR); University of Pittsburgh -- of the Commonwealth System of Higher Education, Pittsburgh, PA (US)

(72) Inventors: Joonho Chung, Seoul (KR); Youngjae Chun, Pittsburgh, PA (US)

(21) Appl. No.: 15/371,339

(22) Filed: Dec. 7, 2016

(30) Foreign Application Priority Data
Dec. 9, 2015 (KR) 10-2015-0174954

(51) Int. Cl. A61F 2/06; A61F 2/88; A61F 2/86

(52) U.S. Cl. CPC A61F 2/06; A61F 2/86; A61F 2/88; A61F 2002/068

(57) ABSTRACT

The present invention relates to a device for changing a course of a vessel, a method of introducing the device, a method of changing a course of a vessel using the device, and a treatment method of brain-nervous system diseases, such as neurovascular compression syndrome, using the device. In treating a neurovascular compression syndrome, the present invention can reduce risks and side effects of the microsurgery (MVD), such as hearing loss or facial paralysis that may occur due to the microsurgery. Further, since an area of a metal surface is small, a risk of in-stent stenosis or thrombus generation is low even in a vessel of the brain having a small diameter, and a degree of vascular compression by its radial force is low, thereby making it possible to minimize intimal hyperplasia.

### BACKGROUND OF THE INVENTION

Field of the Invention

Exemplary embodiments of the present invention relate to a device for changing a course of a vessel, a method of introducing the device for changing a course of a vessel into the vessel, a method of changing a course of a vessel using the device for changing a course of a vessel, and a treatment method of brain-nervous system diseases using the device for changing a course of a vessel.

Description of the Related Art

A neurovascular compression syndrome is a disease caused when a vessel compresses a nerve, and typically includes trigeminal neuralgia and hemifacial spasm.

Trigeminal neuralgia is a facial pain syndrome in which severe electrical and lancinating pain occurs in one or more branches of the trigeminal nerve, and is cranial neuralgia causing facial pain mainly due to compression of the origin of the trigeminal nerve (fifth cranial nerve) by a vessel of the brain.

Further, hemifacial spasm, one of typical neurovascular compression syndromes, is a disease causing repetitive involuntary contraction of hemifacial muscles due to compression of the origin of a facial nerve (seventh cranial nerve) by a vessel of the brain.

The most perfect treatment method for these two diseases is a surgery of separating a vessel of the brain compressing the trigeminal nerve or the facial nerve from these nerves. This surgery is a brain surgery called microvascular decompression (MVD), and is a treatment method of relieving stimulation to a cranial nerve by surgically opening the skull at behind the ear and inserting a teflon between the compressing brain vessel and the compressed nerve (Department of Neurosurgery, University of Cincinnati (www.mayfieldclinic.com/PE-MVD.htm)).

However, in the case of the MVD, a risk accompanying surgery is known as about 2 to 3%, and various disorders such as hearing loss, strabismus, facial paralysis, hoarseness, difficulty swallowing, etc. may occur due to cranial nerve damage, and in severe cases, severe swelling of the brain may be caused leading to death. As a result of surveying patients underwent craniotomy like MVD for their neurovascular compression syndrome, most patients answered that if there is any other perfect treatment method other than craniotomy (on the assumption that risks are equivalent), they would choose the treatment, as such, surgically opening the skull (craniotomy) puts great pressure on patients and their family.

Meanwhile, recently, both of an ischemic cerebrovascular disease caused by atherosclerosis, etc., and a hemorrhagic cerebrovascular disease such as intracranial aneurysm, cerebrovascular malformation, etc. are treated by using endovascular treatment, similarly to a vessel of the heart. Up to now, intracranial endovascular stents have been developed for treating cerebrovascular diseases, and mainly used for four diseases. The intracranial endovascular stents include, first, a stent for intracranial aneurysm for preventing a coil from protruding into a normal vessel during intracranial aneurysm embolization, and second, a flow diverter by which intracranial aneurysm is obliterated by a thrombus through a hemodynamic change. The intracranial endovascular stents further include a stent for cerebrovascular stenosis for widening a narrowed vessel of the brain, and lastly, a stentriever for removing a thrombus blocking a vessel in the case of acute infarction.

These stents are characterized in that most of them have been developed to be used for vascular diseases.

However, an intracranial endovascular treatment device capable of replacing the existing craniotomy by changing only a course of a vessel while maintaining functions of a normal vessel is hardly known.

### SUMMARY OF THE INVENTION

An object of the present invention is to provide a device for changing a course of a vessel.

Another object of the preset invention is to provide a method of introducing the device for changing a course of a vessel into the vessel according to the present invention.

Still another object of the preset invention is to provide a method of changing a course of a vessel using the device for changing a course of a vessel according to the present invention.

Still yet another object of the preset invention is to provide a treatment method of brain-nervous system diseases using the device for changing a course of a vessel according to the present invention.

However, objects of the present invention are not limited to the above-mentioned objects. Other objects that are not mentioned above could be obviously understood by those skilled in the art to which the present invention pertains from the following description.

In accordance with one aspect of the present invention, a device for changing a course of a vessel includes: a fixed part configured to be elastically expandable to support a vessel wall; and an extension part extending by a predetermined length from the fixed part to change the course of the vessel with its elasticity in longitudinal direction.

The device may further include a connecting part that connects the fixed part with the extension part.

The number of fixed part may be two or more.

The fixed part may have a ring shape in which a top dead center and a bottom dead center are continuously formed.

The extension part may be connected to the top dead center, the bottom dead center, or any one point of a line connecting the top dead center and the bottom dead center to each other.

The extension part may be formed by an open-type wire.

The extension part may have a straight line shape, a spiral shape, or a curved line shape.

The device may further include a spherical cap or a bent part at one distal end of the extension part.

The number of extension part may be one or two or more.

The extension part may include a first extension part extending by a predetermined length from the fixed part toward the inflow side of the vessel, and a second extension part extending by a predetermined length from the fixed part toward the outflow side of the vessel.

The device may further include a first connecting part connecting any one point of the fixed part and the first extension part to each other, and a second connecting part connecting any one point of the fixed part and the second extension part to each other.

The number of the first extension part and the number of the second extension part may be one or two or more, respectively.

The first extension part may include a 1-1-th extension part extending by a predetermined length from any one point of the fixed part, and a 1-2-th extension part extending by a predetermined length from any one point of the fixed part.

The extension part may be formed by a closed-type wire.

The extension part may further include a 1-1-th extension part extending by a predetermined length from a 1-1-th connecting part connected to any one point of the fixed part toward an inflow side of the vessel, a 1-2-th extension part extending by a predetermined length from a 1-2-th connecting part connected to any one point of the fixed part toward the inflow side of the vessel, wherein the device further comprising a first body part connecting both distal ends of the 1-1-th extension part and the 1-2-th extension part to each other.

The device may further include a first body connecting part connecting one distal end of the first body part and the 1-1-th extension part to each other, and a second body connecting part connecting the other distal end of the first body part and the 1-2-th extension part to each other.

The number of fixed part may be two or more, and any two fixed parts among a plurality of fixed parts may be connected by the extension part.

The device may further include a connecting part connected to the fixed part at both distal ends of the extension part.

The fixed part may include a first fixed part, and a second fixed part installed to be spaced apart from the first fixed part while facing the first fixed part.

The device may further include a third connecting part extending by a predetermined length from any one point of the first fixed part, and a fourth connecting part extending by a predetermined length from any one point of the second fixed part, in which one distal end of the third connecting part and one distal end of the fourth connecting part are connected to the extension part extending by the predetermined length.

The device may further include a first coupling part connecting one distal end of the third connecting part and one end of the extension part to each other, and a second coupling part connecting one distal end of the fourth connecting part and the other end of the extension part to each other.

The first coupling part and the second coupling part may have a curved line shape.

The extension part may have a straight line shape, a curved line shape, a spiral shape, or an “S”-letter shape.

In accordance with another aspect of the present invention, a method of introducing a device for changing a course of a vessel, includes: compressing the device according to the present invention; introducing the compressed device into a vessel of the brain of a target object; and expanding the introduced device in a length direction of the vessel.

In accordance with another aspect of the present invention, a method for changing a course of a vessel using a device for changing a course of a vessel includes: compressing the device according to the present invention; introducing the compressed device into a vessel of a target object of which a course is to be changed;

and expanding the introduced device in a length direction of the vessel.

In accordance with another aspect of the present invention, a treatment method of a neurovascular compression syndrome includes: compressing the device according to the present invention; introducing the compressed device into a vessel of the brain of a target object; and expanding the introduced device in a length direction of the vessel after the introducing.

The device may be introduced into a central portion or a proximal portion of the vessel compressing a nerve.

The treatment method of the present invention may be to treat a neurovascular compression syndrome compressing a nerve, by changing a course of a vessel compressing the nerve.

The neurovascular compression syndrome may be a trigeminal nerve compression syndrome or a facial nerve compression syndrome, and the trigeminal nerve compression syndrome or the facial nerve compression syndrome may be one or more selected from the group consisting of trigeminal neuralgia, blepharospasm, neurologic pain, hemifacial spasm, and headache.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates a device for changing a course of a vessel according to an exemplary embodiment of the present invention.

FIG. 2 illustrates a device for changing a course of a vessel according to an exemplary embodiment of the present invention.

FIG. 3 illustrates a device for changing a course of a vessel according to an exemplary embodiment of the present invention.

FIG. 4 illustrates a device for changing a course of a vessel according to an exemplary embodiment of the present invention.

FIG. 5 illustrates a device for changing a course of a vessel according to an exemplary embodiment of the present invention.

FIG. 6 illustrates a device for changing a course of a vessel according to an exemplary embodiment of the present invention.

FIG. 7 illustrates a device for changing a course of a vessel according to an exemplary embodiment of the present invention.

FIG. 8 illustrates a device for changing a course of a vessel according to an exemplary embodiment of the present invention.

FIG. 9 illustrates a device for changing a course of a vessel according to an exemplary embodiment of the present invention.

FIG. 10 illustrates a device for changing a course of a vessel according to an exemplary embodiment of the present invention.

FIG. 11 illustrates a device for changing a course of a vessel according to an exemplary embodiment of the present invention.

FIG. 12 illustrates a device for changing a course of a vessel according to an exemplary embodiment of the present invention.

FIG. 13 illustrates a device for changing a course of a vessel according to an exemplary embodiment of the present invention.

FIG. 14 illustrates a device for changing a course of a vessel according to an exemplary embodiment of the present invention.

FIG. 15 illustrates a device for changing a course of a vessel according to an exemplary embodiment of the present invention.

FIG. 16 illustrates a device for changing a course of a vessel according to an exemplary embodiment of the present invention.

FIG. 17 illustrates a device for changing a course of a vessel according to an exemplary embodiment of the present invention.

FIG. 18 illustrates a device for changing a course of a vessel according to an exemplary embodiment of the present invention.

FIG. 19 illustrates a device for changing a course of a vessel according to an exemplary embodiment of the present invention.

FIG. 20 illustrates a device for changing a course of a vessel according to an exemplary embodiment of the present invention.

FIG. 21 illustrates a device for changing a course of a vessel according to an exemplary embodiment of the present invention.

### DESCRIPTION OF SPECIFIC EMBODIMENTS

Hereinafter, a device for changing a course of a vessel according to the present invention will be described in detail with reference to the accompanying drawings, and the exemplary embodiments to be described blow are only to more specifically describe the present invention, thus the scope of the present invention is not limited to the exemplary embodiments.

Conventionally, a stent has been mainly used to widen the lumen of organs such as esophagus that is narrowed due to diseases like gastric and esophageal cancer or other stenosis, or the lumen of vessels. Therefore, a stent focusing on a radial force inside a vessel for expanding the vessel has been developed.

Meanwhile, in a case of neurovascular compression syndrome occurring due to an abnormal course of a vessel of the brain, craniotomy has been generally performed for its treatment. However, in the case of performing the craniotomy, various disorders such as hearing loss, strabismus, facial paralysis, hoarseness, difficulty swallowing, etc. may occur due to cranial nerve damage, and in severe cases, severe swelling of the brain may be caused leading to death.

The present invention provides a device for changing a course of a vessel capable of replacing the existing craniotomy by changing only the course of the vessel while maintaining functions of a normal vessel, in particular, a vessel of the brain.

A device for changing a course of a vessel according to the present invention may include a fixed part and an extension part.

In the present invention, the fixed part may be configured to be elastically expandable to support a vessel wall. In the present invention, the fixed part may be docked on any one point of the vessel, and may also additionally perform a function of expanding an inner wall of the vessel.

Generally, unlike other vessels, a vessel of the brain has a diameter of about 1 mm to 5 mm, thus it is preferable that the fixed part has a diameter of 0.8 to 4.5 mm so that the fixed part may be introduced into the vessel of the brain.

The number of fixed parts is not particularly limited in the present invention, but may be one or two or more.

In the present invention, the extension part functions to change the course of the vessel to a desired direction. The extension part may extend by a predetermined length toward an inflow side of the vessel or an outflow side of the vessel based on a flow direction of the blood flow. A specific shape of the extension part is not particularly limited, but the extension part may be, for example, an open-type wire or a closed-type wire, more specifically, a straight wire, a spiral wire, or a curved wire. However, the curved wire is preferred since a pressure applied to the vessel wall may be minimized.

Further, the wire is not particularly limited in the present invention, but may be shape-memory alloy. It is preferable that the shape-memory alloy is nickel-titanium alloy known as nitinol that is manufactured by cold drawing process, stainless steel, Zninc-Cobalt-Chromium Alloy or tantlum.

Further, the number of extension parts is not particularly limited in the present invention, but may be one or two or more, and may be four, but is not limited in number.

In the present invention, the extension part is to change the course of the vessel to a desired direction, thus a length thereof is preferably 2.4 to 13.5 mm or less, but is not limited thereto.

In the present invention, the extension part may be directly connected to the fixed part, but it is more preferable that the connection is made through a connecting part connecting any one point of the fixed part and any one distal end of the extension part.

In the present invention, the connecting part may be formed to have a predetermined length, and a specific shape thereof is not particularly limited, but may be, for example, a straight line shape, a curved line shape, or a spiral shape.

However, the curved line shape is preferred since a pressure that may be applied to the vessel wall may be minimized while smoothing the connection between the fixed part and the extension part.

Further, the number of connecting parts may be dependent on the number of extension parts in the present invention.

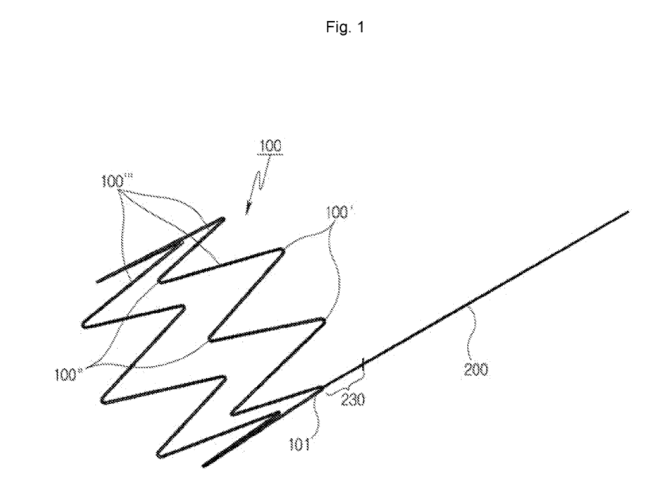

FIGS. 1 to 13 illustrate an exemplary structure of a device for changing a course of a vessel including a fixed part 100 and an extension part 200 according to an exemplary embodiment of the present invention.

As shown in FIG. 1, in the present invention, the fixed part 100 is inserted into and installed in a vessel, that is, a vessel of the brain to determine a position where the device according to the present invention is to be installed. A specific shape of the fixed part 100 is not particularly limited, but may preferably be a ring shape to be matched to a cross section of the vessel having a circular shape. More preferably, the fixed part 100 may have a ring shape in which a top dead center 100′ and a bottom dead center 100″ are continuously formed so that a cross-sectional area of the fixed part may be contracted or expanded depending on a size of an inner wall of the vessel.

Further, in the present invention, a straight line-shaped connecting part 230 having a predetermined length may be connected to any one point 101 of the fixed part 100 to connect the point 101 of the fixed part 100 and one distal end of the extension part 200. In the present invention, the extension part 200 may extend by a predetermined length toward an inflow side or an outflow side of the vessel while being connected to the other distal end of the connecting part 230.

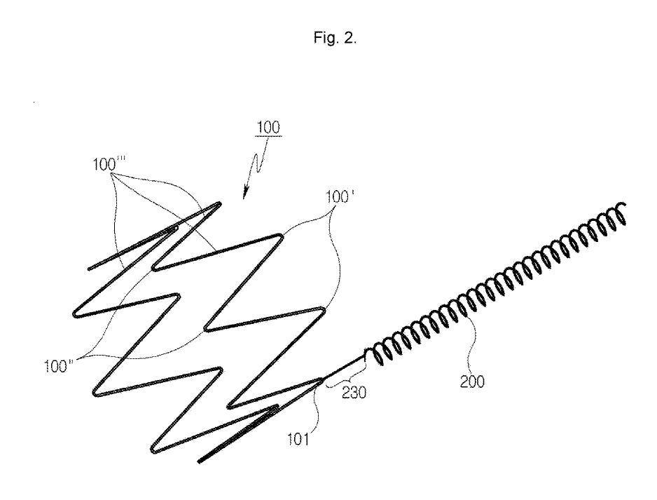

Further, in the present invention, a specific shape of the extension part 200 is not particularly limited, but as shown in FIG. 1, may be a straight line shape, and as shown in FIG. 2, may be a spiral shape or a curved line shape. It is preferable that the extension part 200 has a curved line shape in order to minimize a pressure directly applied to the vessel, in particular, a vessel of the brain having a relatively small diameter.

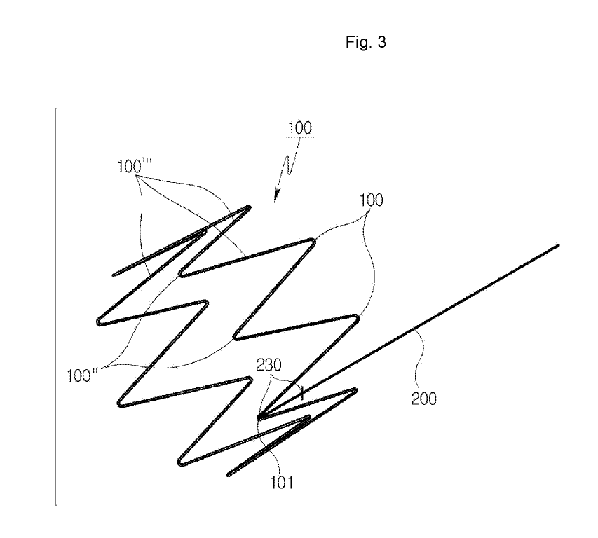

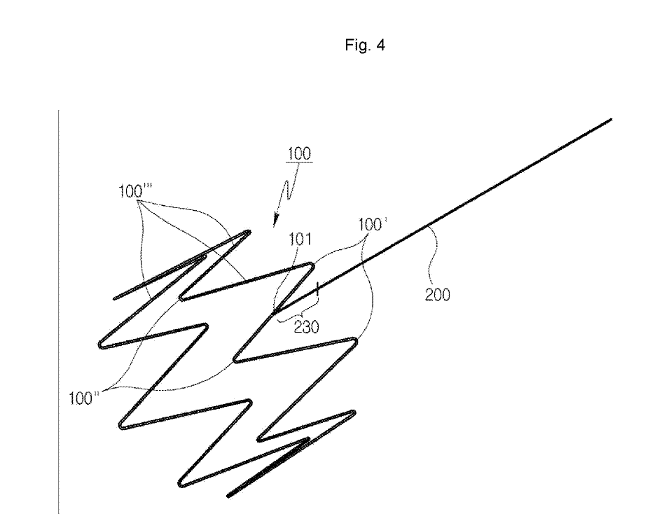

In the present invention, any one point 101 of the fixed part 100 to which the connecting part 230 is connected may be any one point of an outer circumferential surface forming the ring-shaped fixed part 100. A specific position thereof is not particularly limited, however, for example, any one point 101 of the fixed part 100 to which the connecting part 230 is connected may be any one point 101 present at the top dead center 100′ of the outer circumferential surface forming the ring-shaped fixed part 100 as shown in FIGS. 1 and 2, may be any one point 101 present at the bottom dead center 100″ as shown in FIG. 3, or may be any one point 101 present at any one point 100′ of an arbitrary line connecting the top dead center 100′ and the bottom dead center 100″ to each other as shown in FIG. 4.

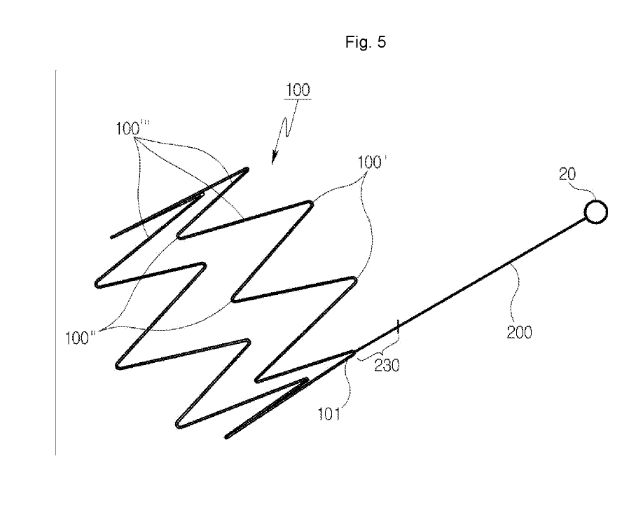

As shown in FIG. 5, in the present invention, in order to minimize the pressure applied to the vessel wall, the extension part 200 further includes a spherical cap 20 thereby minimizing the pressure applied to the vessel wall by a distal end of the extension part 200, and simplifying the configuration of the device, such that side effects such as thrombus generation that may occur in the vessel may be prevented.

Further, in the present invention, a bent part may be further included at one distal end of the extension part, so that one distal end of the extension part is not exposed to the inner wall of the vessel, thereby minimizing the pressure applied to the brain vessel wall by the distal end of the extension part, and simplifying the configuration of the device, such that side effects such as thrombus generation that may occur in the vessel may be prevented.

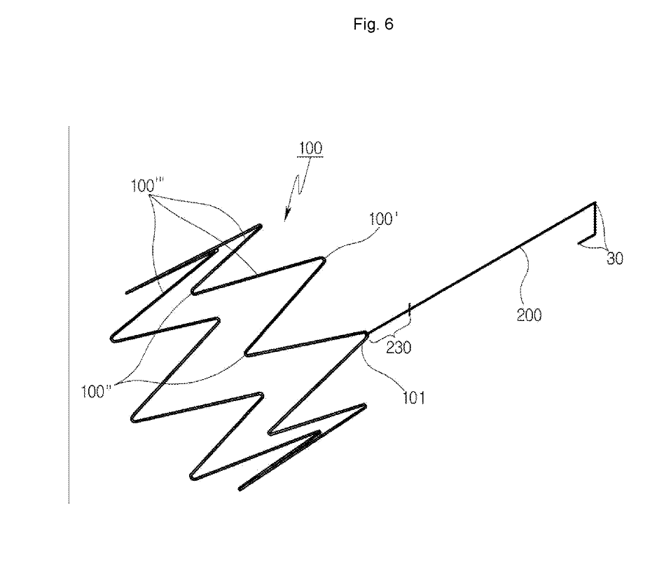

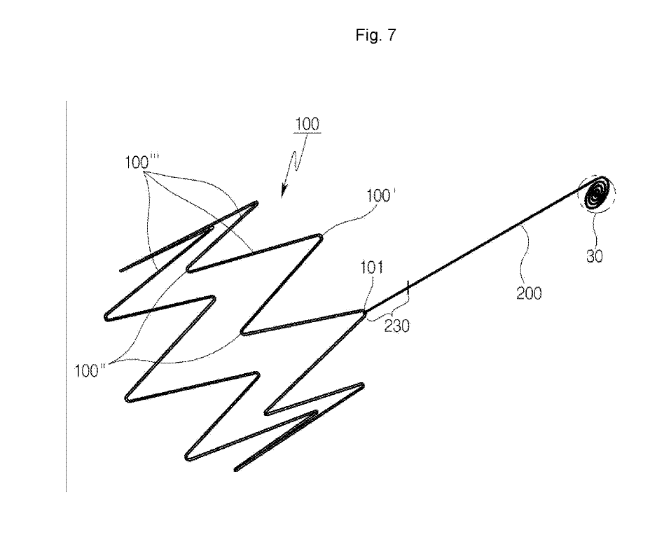

In the present invention, a shape of the bent part is not particularly limited as long as it is a structure in which one distal end of the extension part is not exposed to the brain vessel wall. For example, the bent part 30 may have ‘L’-letter shape as shown in FIG. 6, or have a spirally wound shape as shown in FIG. 7.

As described above, in the present invention, one or two or more extension parts 200 may be included. However, it is preferable that two or more extension parts 200 are included in order to stably change the course of the vessel.

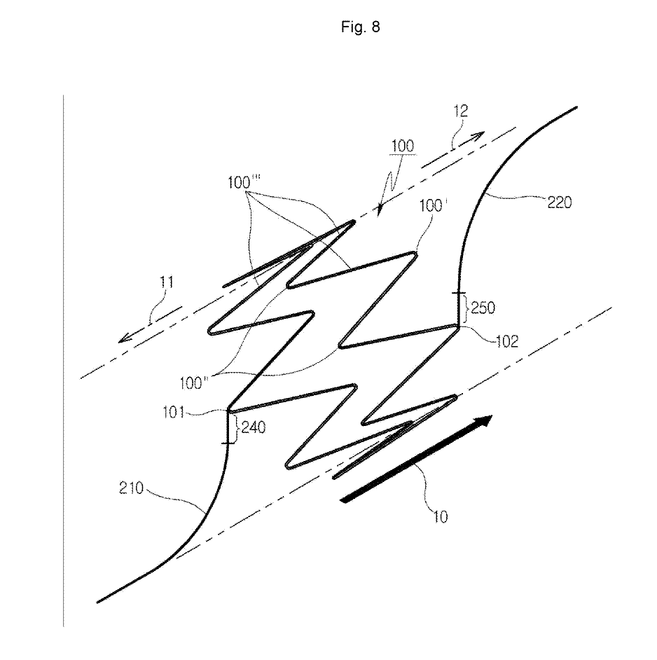

As shown in FIG. 8, in an exemplary embodiment of the present invention, the extension part may include a first extension part 210 extending by a predetermined length from any one point 101 of the fixed part 100 through a first connecting part 240 against a direction in which blood is introduced based on a blood flow direction 10, that is, toward an inflow side of the vessel 11, and a second extension part 220 extending by a predetermined length from any one point 102 of the fixed part 100 through a second connecting part 250 against a direction in which blood is discharged, that is, toward an outflow side of the vessel 12. Here, the first extension part 210 and the second extension part 220 may be formed to be inclined to support one side or the other side of the inner wall of the vessel.

In the present invention, any one point 101 of the fixed part 100 to which the first connecting part 240 is connected may be the same as or different from the other one point 102 of the fixed part 100 to which the second connecting part 250 is connected, and is not particularly limited.

However, in the present invention, any one point 101 of the fixed part 100 to which the first connecting part 240 is connected is formed at a farthest point of a circle from the other one point 102 of the fixed part 100 to which the second connecting part 250 is connected, which is more stable since the first extension part 210 may support the vessel of the inflow side of the vessel 11 and the second extension part 220 may support the vessel of the outflow side of the vessel 12 thereby changing the course of the vessel in a balanced way.

In the present invention, “the farthest point of the circle” means a point of the fixed part 100 positioned at a farthest distance from a connection starting point of the first connecting part 240, when the fixed part 100 has a circular shape, and may mean a point 102 in which a length of a chord connecting between the connection starting point of the first connecting part 240 and the other point 102 of the outer circumferential surface of the fixed part becomes longest.

Further, in the present invention, as described above, the number of the first extension part extending from the fixed part or the first connecting part toward the inflow side of the vessel may be one or two or more, preferably, two or more. As an example of the present invention, the first connecting part may include a 1-1-th connecting part and a 1-2-th connecting part, and the first extension part may include a 1-1-th extension part and a 1-2-th extension part, but not limited thereto.

Further, in the present invention, the number of the second extension part extending from the fixed part or the second connecting part toward the outflow side of the vessel may be one or two or more, preferably, two or more. As an example of the present invention, the second connecting part may include a 2-1-th connecting part and a 2-2-th connecting part, and the second extension part may include a 2-1-th extension part and a 2-2-th extension part, but not limited thereto.

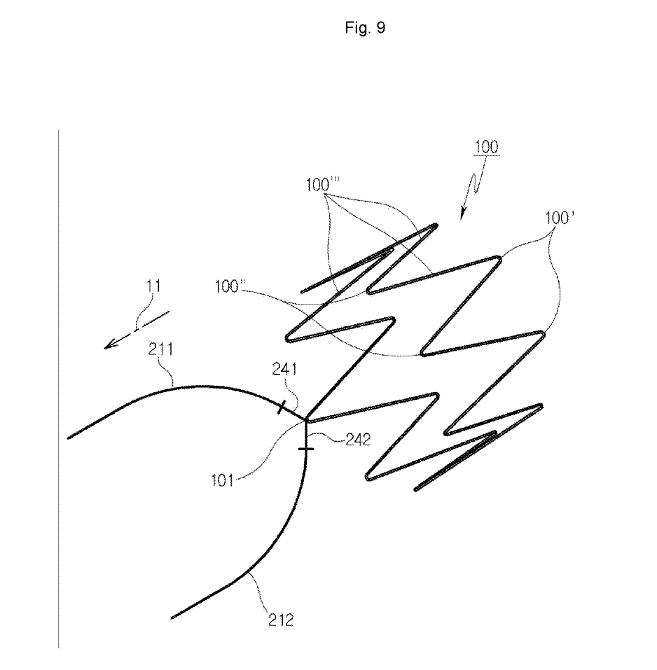

As shown in FIG. 9, in an exemplary embodiment of the present invention, the extension part may include a 1-1-th extension part 211 extending by a predetermined length from any one point 101 of the fixed part 100 through a 1-1-th connecting part 241 toward the inflow side of the vessel 11, and a 1-2-th extension part 212 extending by a predetermined length from the same one point 101 of the fixed part 100 through a 1-2-th connecting part 242 toward the inflow side of the vessel 11. Here, the 1-1-th extension part 211 may be formed to be inclined to support one side of the inner wall of the vessel, and the 1-2-th extension part 212 may be formed to be inclined to support the other side of the inner wall of the vessel.

In addition, although not illustrated, in an exemplary embodiment of the present invention, the extension part may include a 2-1-th extension part extending by a predetermined length from any one point of the fixed part through a 2-1-th connecting part toward the outflow side of the vessel, and a 2-2-th extension part extending by a predetermined length from the same one point of the fixed part through a 2-1-th connecting part toward the outflow side of the vessel. Here, the 2-1-th extension part may extend to be inclined to support one side of the inner wall of the vessel, and the 2-2-th extension part may be formed to be inclined to support the other side of the inner wall of the vessel.

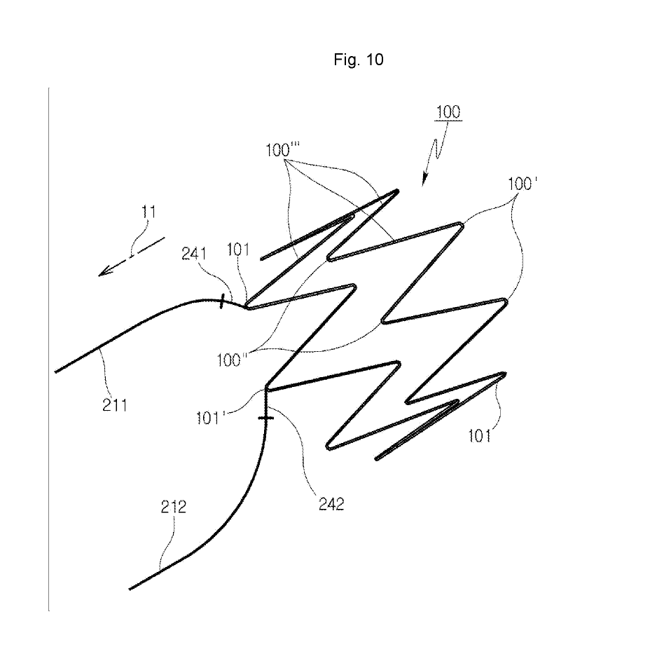

As shown in FIG. 10, in an exemplary embodiment of the present invention, the extension part may include a 1-1-th extension part 211 extending by a predetermined length from any one point 101 of the fixed part 100 through a 1-1-th connecting part 241 toward the inflow side of the vessel 11, and a 1-2-th extension part 212 extending by a predetermined length from the other one point 101′ positioned to be spaced apart from the any one point 101 of the fixed part 100 through a 1-2-th connecting part 242 toward the inflow side of the vessel 11. Here, the 1-1-th extension part 211 may extend, by a predetermined length, to be inclined to support one side of the inner wall of the vessel, and the 1-2-th extension part 212 may be formed to be inclined to support the other side of the inner wall of the vessel.

In addition, although not illustrated, in an exemplary embodiment of the present invention, the extension part may include a 2-1-th extension part extending by a predetermined length from any one point of the fixed part through a 2-1-th connecting part toward the outflow side of the vessel, and a 2-2-th extension part extending by a predetermined length from the other one point positioned to be spaced apart from the any one point of the fixed part toward the outflow side of the vessel.

Here, the 2-1-th extension part may extend, by a predetermined length, to be inclined to support one side of the inner wall of the vessel, and the 2-2-th extension part may be formed to be inclined to support the other side of the inner wall of the vessel.

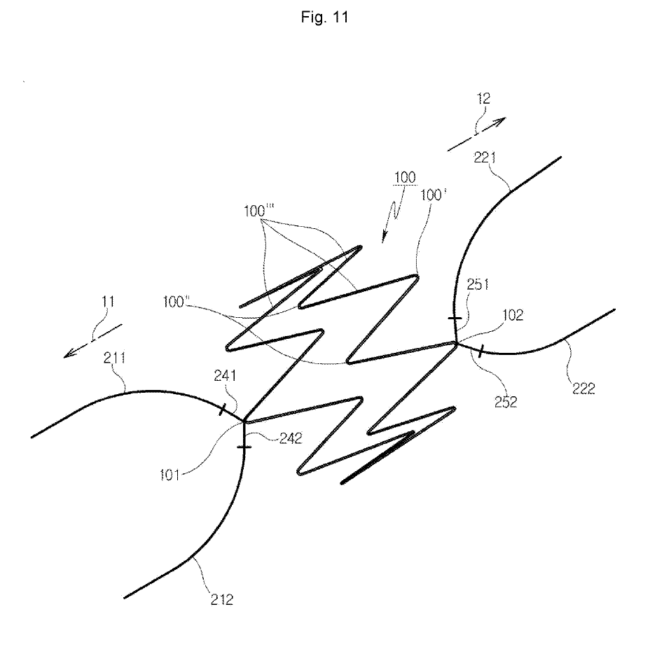

As shown in FIG. 11, in an exemplary embodiment of the present invention, the extension part may include a 1-1-th extension part 211 extending by a predetermined length from any one point 101 of the fixed part 100 through a 1-1-th connecting part 241 toward the inflow side of the vessel 11, and a 1-2-th extension part 212 extending by a predetermined length from the same one point 101 of the fixed part 100 through a 1-2-th connecting part 242 toward the inflow side of the vessel 11. In addition, the extension part may include a 2-1-th extension part 221 extending by a predetermined length from a farthest point 102 of a circle from any one point 101 of the fixed part 100 through a 2-1-th connecting part 251 toward the outflow side of the vessel 12, and a 2-2-th extension part 222 extending by a predetermined length from the same point 102 of the fixed part 100 through a 2-2-th connecting part 252 toward the outflow side of the vessel 12. Here, the 1-1-th extension part 211 and the 2-1-th extension part 221 may extend, by a predetermined length, to be inclined to support one side of the inner wall of the vessel, and the 1-2-th extension part 212 and the 2-2-th extension part 222 may extend, by a predetermined length, to be inclined to support the other side of the inner wall of the vessel.

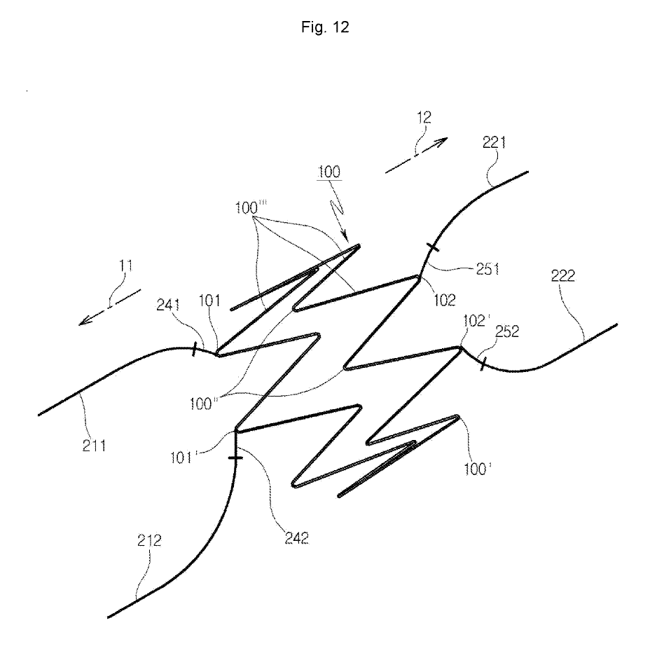

As shown in FIG. 12, in an exemplary embodiment of the present invention, the extension part may include a 1-1-th extension part 211 extending by a predetermined length from any one point 101 of the fixed part 100 through a 1-1-th connecting part 241 toward the inflow side of the vessel 11, and a 1-2-th extension part 212 extending by a predetermined length from the other one point 101′ positioned to be spaced apart from any one point 101 of the fixed part 100 through a 1-2-th connecting part 242 toward the inflow side of the vessel 11, and may include a 2-1-th extension part 221 extending by a predetermined length from a farthest point 102 of a circle from any one point 101 of the fixed part 100 through a 2-1-th connecting part 251 toward the outflow side of the vessel 12, and a 2-2-th extension part 222 extending by a predetermined length from a farthest point 102′ of a circle from the other one point 101′ through a 2-2-th connecting part 252 toward the outflow side of the vessel 12. Here, the 1-1-th extension part 211 and the 2-1-th extension part 221 may extend, by a predetermined length, to be inclined to support one side of the inner wall of the vessel, and the 1-2-th extension part 212 and the 2-2-th extension part 222 may extend, by a predetermined length, to be inclined to support the other side of the inner wall of the vessel.

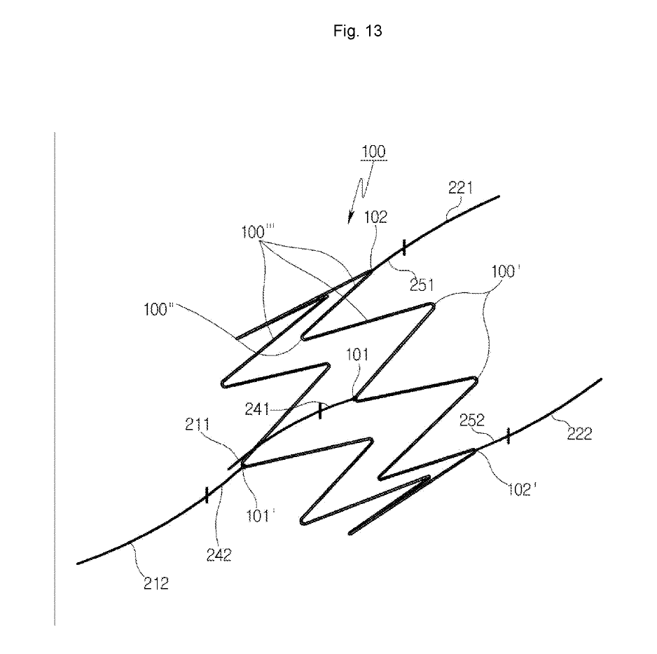

As shown in FIG. 13, in an exemplary embodiment of the present invention, the extension part may include a 1-1-th extension part 211 extending by a predetermined length from any one point 101 of the fixed part 100 through a 1-1-th connecting part 241 toward the inflow side of the vessel 11, and a 1-2-th extension part 212 extending by a predetermined length from a farthest point 101′ of a circle from any one point 101 of the fixed part 100 through a 1-2-th connecting part 251 toward the inflow side of the vessel 11, and may include a 2-1-th extension part 221 extending by a predetermined length from any one point 102 of two points of the outer circumferential surface of the fixed part 100 that contact a straight line perpendicular to a straight line connecting the any one point 101 and the other one point 101′ to each other through a 2-1-th connecting part 251 toward the outflow side of the vessel 12, and a 2-2-th extension part 222 extending by a predetermined length from the other one point 102′ of the two points through a 2-2-th connecting part 252 toward the outflow side of the vessel 12. Here, the 1-1-th extension part 211 and the 2-1-th extension part 221 may extend, by a predetermined length, to be inclined to support one side of the inner wall of the vessel, and the 1-2-th extension part 212 and the 2-2-th extension part 222 may extend, by a predetermined length, to be inclined to support the other side of the inner wall of the vessel.

As described above, when each of four extension parts extends by a predetermined length by being connected from a farthest point of a circle, four side surfaces may be supported, thereby making it possible to more effectively change the course of the vessel.

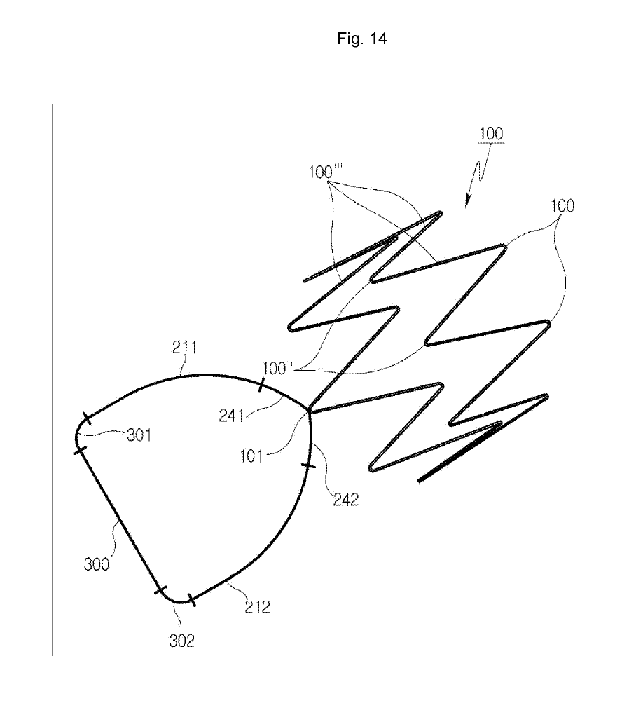

FIGS. 14 to 16 illustrate an exemplary structure of a device for changing a course of a vessel including a fixed part 100 and an extension part 200 according to another exemplary embodiment of the present invention.

As shown in FIG. 14, in an exemplary embodiment of the present invention, a device for changing a course of a vessel may include a fixed part 100, a 1-1-th extension part 211 extending by a predetermined length from any one point 101 of the fixed part 100 through a 1-1-th connecting part 241 toward the inflow side of the vessel 11, and a 1-2-th extension part 212 extending by a predetermined length from the same point 101 of the fixed part 100 through a 1-2-th connecting part 242 toward the inflow side of the vessel 11, in which the other distal end of the 1-1-th extension part 211 and the other distal end of the 1-2-th extension part 212 may be connected by a first body part 300 to form a closed shape. At this point, the 1-1-th extension part 211 and the first body part 300 may be connected by a first body connecting part 301, and the 1-2-th extension part 212 and the first body part 300 may be connected by a second body connecting part 302. Here, it is preferable that the first body connecting part 301 and the second body connecting part 302 have a curved line shape, to minimize a pressure applied to the inner wall of the vessel.

Although not illustrated, in an exemplary embodiment of the present invention, the device for changing a course of a vessel may include a fixed part, a 2-1-th extension part extending by a predetermined length from any one point of the fixed part through a 2-1-th connecting part toward the outflow side of the vessel, and a 2-2-th extension part extending by a predetermined length from the same one point as the any one point of the fixed part through a 2-2-th connecting part toward the outflow side of the vessel, in which a distal end of the 2-1-th extension part and a distal end of the 2-2-th extension part may be connected by a second body part to form a closed shape. At this point, the 2-1-th extension part and the second body part may be connected by a third body connecting part, and the 2-2-th extension part and the second body part may be connected by a fourth body connecting part. Here, it is preferable that the third body connecting part and the fourth body connecting part have a curved line shape, to minimize a pressure applied to the inner wall of the vessel.

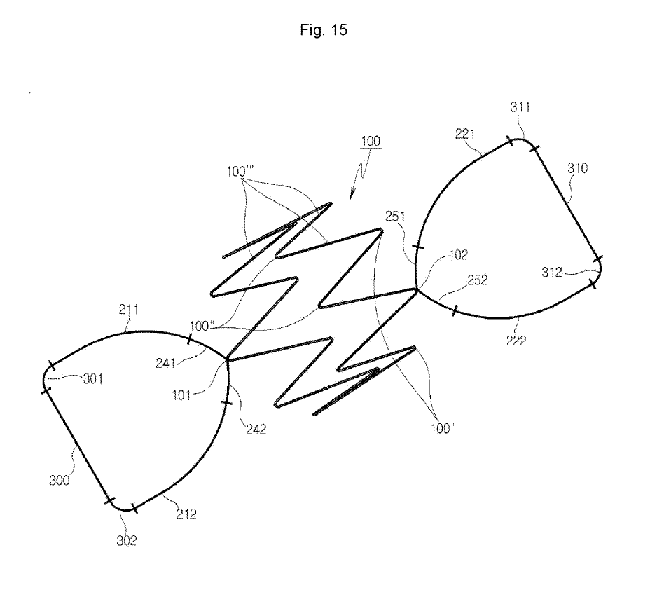

As shown in FIG. 15, in an exemplary embodiment of the present invention, the extension part may include a 1-1-th extension part 211 extending by a predetermined length from any one point 101 of the fixed part 100 through a 1-1-th connecting part 241 of a circle toward the inflow side of the vessel 11, and a 1-2-th extension part 212 extending by a predetermined length from any one point 101 of the fixed part 100 through a 1-2-th connecting part 242 toward the inflow side of the vessel 11, in which a distal end of the 1-1-th extension part 211 and a distal end of the 1-2-th extension part 212 may be connected by a first body part 300 to form a closed shape. At this point, the 1-1-th extension part 211 and the first body part 300 may be connected by a first body connecting part 301, and the 1-2-th extension part 212 and the first body part 300 may be connected by a second body connecting part 302. Further, the extension part may include a 2-1-th extension part 221 extending by a predetermined length from a farthest point 102 of a circle from any one point 101 of the fixed part 100 through a 2-1-th connecting part 251 toward the outflow side of the vessel 12, and a 2-2-th extension part 222 extending by a predetermined length from any one point 102 of the fixed part 100 through a 2-2-th connecting part 252 toward the outflow side of the vessel 12, in which a distal end of the 2-1-th extension part 221 and a distal end of the 2-2-th extension part 222 may be connected by a second body part 310 to form a closed shape. At this point, the 2-1-th extension part 221 and the second body part 310 may be connected by a third body connecting part, and the 2-2-th extension part 222 and the second body part 310 may be connected by a fourth body connecting part 312. Here, it is preferable that the first body connecting part 301, the second body connecting part 302, the third body connecting part 311, and the fourth body connecting part 312 have a curved line shape, to minimize a pressure applied to the inner wall of the vessel. When configured as described above, a portion through which a blood flow of the vessel, in particular, a vessel of the brain having a relatively small diameter moves corresponds to a hollow, not inhibiting the flow of the blood, and a risk of in-stent stenosis or thrombus generation is low.

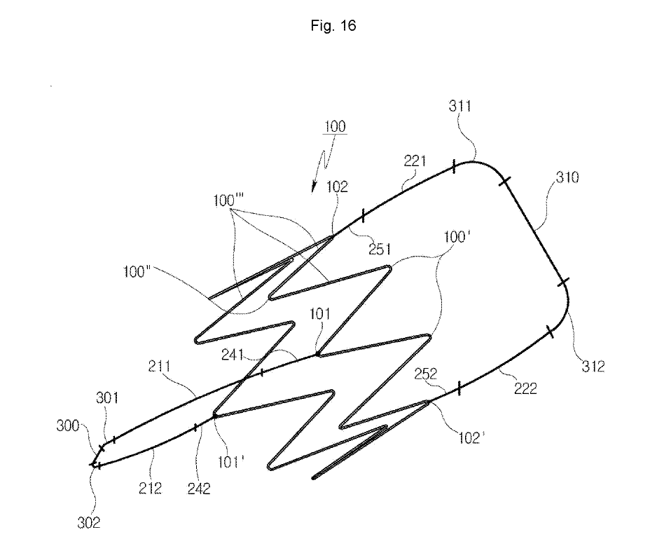

As shown in FIG. 16, in an exemplary embodiment of the present invention, the extension part may include a 1-1-th extension part 211 extending by a predetermined length from any one point 101 of the fixed part 100 through a 1-1-th connecting part 241 toward the inflow side 11 of the vessel, and a 1-2-th extension part 212 extending by a predetermined length from a farthest point 101′ of a circle from the any one point 101 of the fixed part 100 through a 1-2-th connecting part 242 toward the inflow side of the vessel 11, in which a distal end of the 1-1-th extension part 211 and a distal end of the 1-2-th extension part 212 may be connected by a first body part 300 to form a closed shape. At this point, the 1-1-th extension part 211 and the first body part 300 may be connected by a first body connecting part 301, and the 1-2-th extension part 212 and the first body part 300 may be connected by a second body connecting part 302. Further, the extension part may include a 2-1-th extension part 221 extending by a predetermined length from any one point 102 of two points of the outer circumferential surface of the fixed part 100 that contact a straight line perpendicular to a straight line connecting the any one point 101 and the other one point 101′ to each other through a 2-1-th connecting part 251 toward the outflow side of the vessel 12, and a 2-2-th extension part 222 extending by a predetermined length from the other one point 102′ of the two points of the outer circumferential surface through a 2-2-th connecting part 252 toward the outflow side 12 of the vessel, in which a distal end of the 2-1-th extension part 221 and a distal end of the 2-2-th extension part 222 may be connected by a second body part 310 to form a closed shape. At this point, the 2-1-th extension part 221 and the second body part 310 may be connected by a third body connecting part, and the 2-2-th extension part 222 and the second body part 310 may be connected by a fourth body connecting part 312. Here, it is preferable that the first body connecting part 301, the second body connecting part 302, the third body connecting part 311, and the fourth body connecting part 312 have a curved line shape, to minimize a pressure applied to the inner wall of the vessel.

However, in the present invention, the number of the first extension part and the second extension part may be three or more, respectively, and any two of the extension parts extending in the same direction may be connected to the body part through the body connecting parts, and the number of extension parts and the body parts are not limited to the above examples.

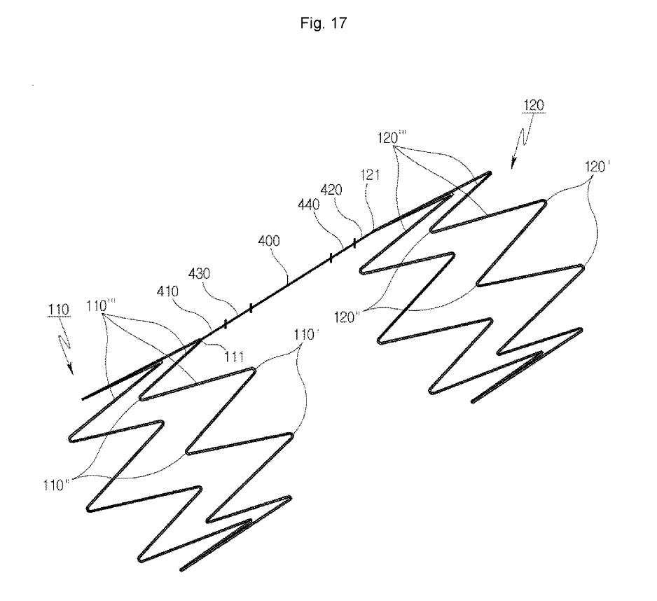

FIGS. 17 to 21 illustrate a device for changing a course of a vessel including a fixed part and an extension part according to another exemplary embodiment of the present invention.

In the present invention, the number of fixed part may be two or more, and as described above, when the number of fixed part is two or more, it is possible to more efficiently support the vessel to effectively change a bent course of the vessel.

Further, in the present invention, the extension part functions to change the course of the vessel to a desired direction. When the number of fixed parts is two or more, the extension part may extend by a predetermined length from any one point of any one fixed part of two fixed parts that are continuously disposed among the two or more fixed parts to any one point of the other one fixed part.

In the present invention, a specific shape of the extension part is not particularly limited, but may be a straight line shape, a curved line shape, a spiral shape, or an “S”-letter shape. When the extension part is bent in the “S”-letter shape, it is possible not to hinder the flow of the blood without separately applying an impact.

In the present invention, both distal ends of the extension part may be directly connected to one point of the two fixed parts, but it is more preferable that both distal ends of the extension part are connected to one point of the two fixed parts through a connecting part. A specific shape of the connecting part is not particularly limited, but may be a straight line shape, a curved line shape, or a spiral shape. It is preferable that the connecting part has a curved line shape, since a pressure applied to the vessel wall may be minimized.

In the present invention, both distal ends of the connecting part may each be directly connected to distal ends of the extension part and any one point of the fixed parts, but it is more preferable that both distal ends of the connecting part are connected through a coupling part. The coupling part may have a straight line shape, a spiral shape, or a curved line shape, but it is preferable that the coupling part has a curved line shape, since the pressure applied to the vessel may be minimized.

As shown in FIG. 17, in an exemplary embodiment of the present invention, the fixed part includes a first fixed part 110, and a second fixed part 120 installed to be spaced apart from the first fixed part 110 and face the first fixed part 110 while having a section of the vessel, in particular, a vessel of the brain of which the course needs to be changed interposed therebetween. The device for changing a course of a vessel includes a third connecting part 410 extending by a predetermined length from any one point 111 of the first fixed part 110 toward the second fixed part 120, and a fourth connecting part 420 extending by a predetermined length from any one point 121 of the second fixed part 120 toward the first fixed part 110, in which the other distal end of the third connecting part 410 and the other distal end of the fourth connecting part 420 may be connected to both ends of the extension part 400. Further, one distal end of the third connecting part 410 may be connected to one distal end of the extension part 400 through a straight line-shaped first coupling part 430, and one distal end of the fourth connecting part 420 may be connected to the other distal end of the extension part 400 through a straight line-shaped second coupling part 440. Here, a shape of the extension part 400 may be a straight line shape, but is not limited thereto.

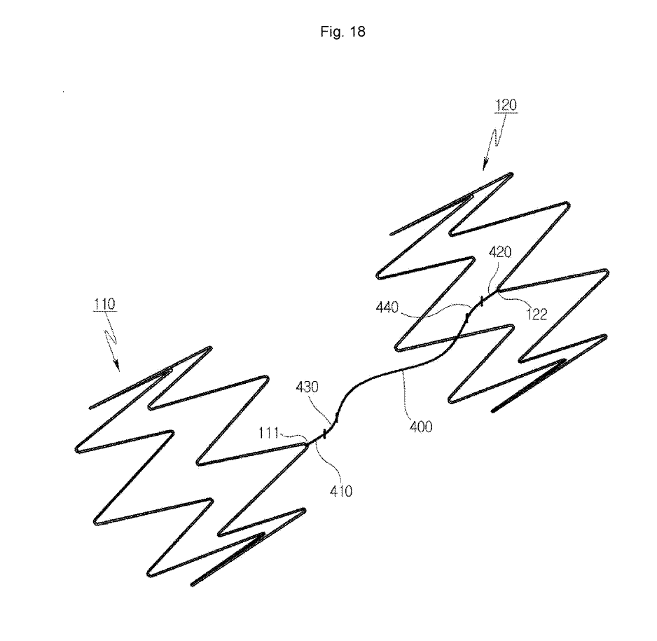

As shown in FIG. 18, in an exemplary embodiment of the present invention, the device for changing a course of a vessel includes a third connecting part 410 extending by a predetermined length from any one point 111 of the first fixed part 110 toward the second fixed part 120, and a fourth connecting part 420 extending by a predetermined length from any one point 122 of the second fixed part 120 toward the first fixed part 110, in which one distal ends of the third and fourth connecting parts 410 and 420 may be connected to both ends of the extension part 400 and one distal end of the third connecting part 410 is connected to one distal end of the extension part 400 through a curved line-shaped first coupling part 430, and one distal end of the fourth connecting part 420 may be connected to the other distal end of the extension part 400 through a curved line-shaped second coupling part 440. Here, a shape of the extension part 400 may be a curved line shape, but is not limited thereto.

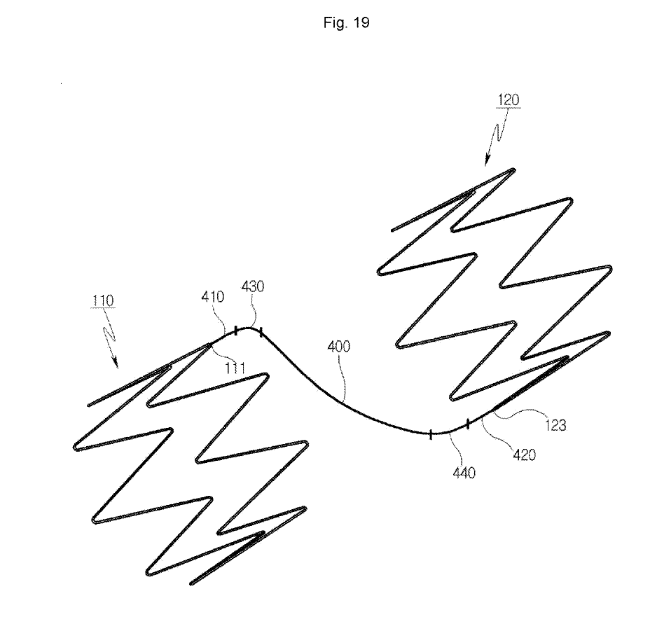

As shown in FIG. 19, in an exemplary embodiment of the present invention, the device for changing a course of a vessel includes a third connecting part 410 extending by a predetermined length from any one point 111 of the first fixed part 110 toward the second fixed part 120, and a fourth connecting part 420 extending by a predetermined length from a farthest point 123 of a circle from a point 122 of the second fixed part 120 corresponding to any one point 111 of the first fixed part 110 toward the first fixed part 110, in which one distal ends of the third and fourth connecting parts 410 and 420 may be connected to both ends of a straight line-shaped extension part 400 and one distal end of the third connecting part 410 is connected to one distal end of the curved line-shaped extension part 400 through a curved line-shaped first coupling part 430, and one distal end of the fourth connecting part 420 may be connected to the other distal end of the extension part 400 through a curved line-shaped second coupling part 440.

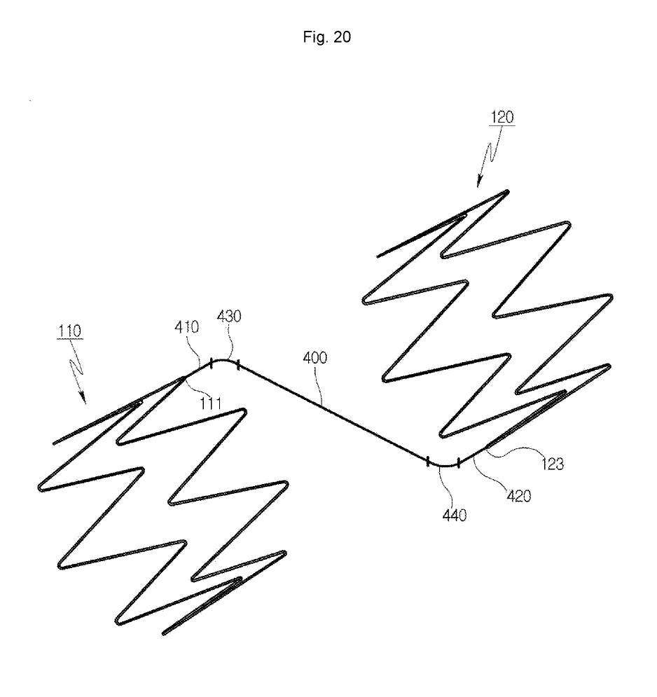

As shown in FIG. 20, in an exemplary embodiment of the present invention, the device for changing a course of a vessel may include a third connecting part 410 extending by a predetermined length from any one point 111 of the first fixed part 110 toward the second fixed part 120, and a fourth connecting part 420 extending by a predetermined length from a farthest point of a circle from a point 122 corresponding to any one point 111 of the first fixed part 110 toward the first fixed part 110. At this point, the other distal end of the third connecting part 410 may be connected to one distal end of a straight line-shaped extension part 400 through a curved line-shaped first coupling part 430, and the other distal end of the fourth connecting part 420 may be connected to the other distal end of the extension part 400 through a curved line-shaped second coupling part 440.

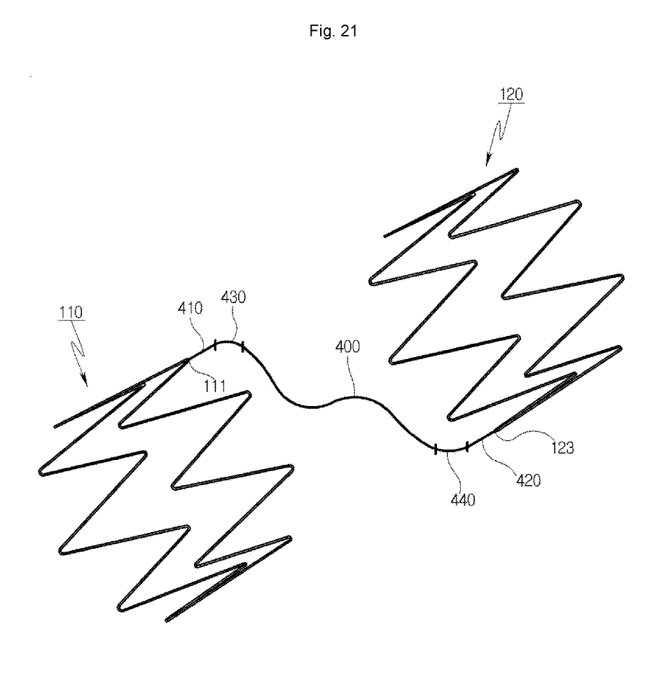

As shown in FIG. 21, in an exemplary embodiment of the present invention, the device for changing a course of a vessel includes a third connecting part 410 extending by a predetermined length from any one point 111 of the first fixed part 110 toward the second fixed part 120, and a fourth connecting part 420 extending by a predetermined length from a farthest point 123 of a circle from a point 122 of the second fixed part 120 corresponding to any one point 111 of the first fixed part 110 toward the first fixed part 110, in which one distal ends of the third and fourth connecting parts 410 and 420 may be connected to both ends of a straight line-shaped extension part 400 and one distal end of the third connecting part 410 is connected to one distal end of the “S”-letter-shaped extension part 400 through a curved line-shaped first coupling part 430, and one distal end of the fourth connecting part 420 may be connected to the other distal end of the extension part 400 through a curved line-shaped second coupling part 440.

Still another exemplary embodiment of the present invention relates to a method of introducing the device for changing a course of a vessel according to the present invention into the vessel. Specifically, the method of introducing the device for changing a course of a vessel into the vessel may include compressing the device for changing a course of a vessel according to the present invention; introducing the compressed device into a vessel of a target object; and expanding the introduced device for changing a course of a vessel in a length direction of the vessel.

In the compressing of the device for changing a course of a vessel according to the present invention, the compression may be performed to an extent that the compressed device may be inserted into a microcatheter, by using a net formed of a biocompatible mesh material capable of compressing the device. However, the method of compressing the device according to the present invention is not limited thereto, and any method for introducing the compressed device in to a vessel of a target object may be included as long as it may compress the flexible device according to the present invention.

The device for changing a course of a vessel according to the present invention may be used in any method, as long as it is a method such as insertion into a microcatheter, excluding craniotomy. As described above, in the case in which the device for changing a course of a vessel according to the present invention is introduced not by craniotomy, there is an advantage in that surgical burden to a patient requiring insertion of the device for changing a course of a vessel into a vessel of the brain, may be reduced.

Still another exemplary embodiment of the present invention relates to a method for changing a course of a vessel using the device according to the present invention. Specifically, the method for changing a course of a vessel using the device may include: compressing the device for changing a course of a vessel according to the present invention; introducing the compressed device into a vessel of a target object of which a course is to be changed; and expanding the introduced device for changing a course of a vessel in a length direction of the vessel.

However, when a material configuring the device according to the present invention is a shape-memory alloy, in the case in which a compressing force from the outside is excluded, due to a characteristic of the material, the expanding in the length direction may be self-expanding.

In the present invention, the wire is not particularly limited in the present invention, but may be shape-memory alloy. It is preferable that the shape-memory alloy is nickel-titanium alloy known as nitinol that is manufactured by cold drawing process, stainless steel, Zninc-Cobalt-Chromium Alloy or tantlum.

Still another exemplary embodiment of the present invention relates to a treatment method of a neurovascular compression syndrome using the device according to the present invention. Specifically, the treatment method of a neurovascular compression syndrome using the device according to the present invention may include: compressing the device for changing a course of a vessel according to the present invention; introducing the compressed device into a vessel of a target object; and expanding the introduced device in a length direction of the vessel after the introducing.

However, the target object of the present invention means a patient with a neurovascular compression syndrome whose cranial nerve is compressed due to an abnormal course of a vessel.

In the compressing of the device according to the present invention, the compression may be performed to an extent that the compressed device may be inserted into a microcatheter. However, the method of compressing the device according to the present invention is not limited thereto, and any method may be included as long as it may compress the flexible device according to the present invention.

The device according to the present invention may be introduced into a central portion or a proximal portion of the vessel compressing the nerve, but is not limited thereto, and may be inserted into any portion, as long as it is a portion of the vessel compressing the nerve.

The treatment method of the present invention is to treat a neurovascular compression syndrome compressing a nerve, by changing a course of a vessel compressing the nerve.

However, “the course of the vessel” in the present invention means a direction in which blood flows in the vessel, and for the purpose of the present invention, means a blood flow direction in a state in which the vessel is abnormally bent to an extent that the vessel compresses the surrounding nerve.

Further, the “neurovascular compression syndrome” in the present invention may occur when inner pulsatile pressure of an intracranial vessel compresses a facial nerve, and in this case, the neurovascular compression syndrome corresponds to hemifacial spasm an abnormal functional nervous disease.

The device according to the present invention is introduced in a normal vessel compressing a cranial nerve such as a trigeminal nerve (fifth cranial nerve) or a facial nerve (seventh cranial nerve) to correct a course of the normal vessel that is bent, such that the compressed cranial nerve is decompressed, thereby making it possible to alleviate or treat the disease.

In the present invention, the “vessel” is a tubule through which blood passes, and for the purpose of the present invention, the vessel may be a vessel of the brain. Specifically, the vessel of the brain may be the superior cerebellar artery (SCA), the anterior inferior cerebellar artery (AICA), the posterior inferior cerebellar artery (PICA) or the vertebral artery (VA), but is not limited thereto.

In the present invention, the superior cerebellar artery (SCA) is a most common causative vessel among vessels causing trigeminal neuralgia, and a diameter thereof is measured as about 1.2 to 2.0 mm. The SCA mainly compresses a trigeminal nerve at a loop of the anterior or lateral pontomesencephalic segment.

Further, in the present invention, the anterior inferior cerebellar artery (AICA) is a most common causative vessel among vessels causing hemifacial spasm, and an average diameter thereof is about 0.5 to 1.5 mm, and the AICA is divided into the “small AICA” (<0.8 mm), the “medium AICA” (0.8˜1.2 mm), and the “strongly developed AICA” “(>1.2 mm)”. The AICA compresses a facial nerve at a loop of the lateral pontine segment. Most causative vessels of hemifacial spasm is known as the AICA, and in this case, considering a characteristic of the AICA, a device for changing a course of a vessel of the brain, having a diameter of 0.5 to 2.5 mm may be used.

Further, in the present invention, a thickness of the posterior inferior cerebellar artery (PICA) is measured as about 1.0 to 1.5 mm. The PICA compresses a facial nerve when the lateral medullary segment or the tonsilomedullary segment is long and tortuous.

Further, in the present invention, the vertebral artery (VA) is severely bent to be biased to one side in rare cases, and in this case, the VA compresses a facial nerve. Generally, an average thickness of the VA is about 2.0 to 3.5 mm, and the VA is one of vessels on which stent insertion surgery for treating cerebrovascular diseases is mainly performed presently, but has not been used for the purpose of treating neurovascular compression syndromes.

In the present invention, the “cranial nerve” is a concept including twelve nerves extending from the brainstem, and includes a trigeminal nerve (fifth cranial nerve), a facial nerve (seventh cranial nerve), an olfactory nerve, an optic nerve, an oculomotor nerve, a trochlear nerve, an abducent nerve, an auditory nerve, a glossopharyngeal nerve, a vagus nerve, a hypoglossal nerve, and an accessory nerve.

The neurovascular compression syndrome of the present invention may be a trigeminal nerve compression syndrome or a facial nerve compression syndrome, and preferably, the trigeminal nerve compression syndrome or the facial nerve compression syndrome may be one or more selected from the group consisting of trigeminal neuralgia, hemifacial spasm, blepharospasm, neurologic pain, and headache.

A treatment of trigeminal neuralgia and hemifacial spasm according to the related art uses a drug treatment (Botox treatment, etc.) as a conservative treatment, or uses the MVD as a radical treatment in most cases. However, the drug treatment is not a radical treatment method, thus merely alleviates symptoms temporarily, and the MVD is craniotomy, thus has a disadvantage in that a risk that a patient and a doctor should take is high.

Accordingly, the treatment method of a neurovascular compression syndrome including introducing the device for changing a course of a vessel according to the present invention into a vessel of a target object, has advantages in that an emotional burden of a patient may be reduced by introducing the device, and a risk of side effects such as hearing loss or facial paralysis that may occur due to craniotomy may be decreased.

In the case of using the device for changing a course of a vessel according to the present invention, in treating a neurovascular compression syndrome, risks of the surgery are lower than when using the existing surgical method, and a risk of side effects such as hearing loss or facial paralysis that may occur due to craniotomy, etc. may be significantly decreased.

Further, in the case of using the device according to the present invention, since an area of a metal surface is small, a risk of in-stent stenosis or thrombus generation is low even in a vessel of the brain having a small diameter, and a degree of vascular compression is low, thereby making it possible to minimize intimal hyperplasia.

1. A device for changing a course of a vessel, the device comprising:
a fixed part configured to be elastically expandable to support a vessel wall; and
an extension part extending by a predetermined length from the fixed part to change the course of the vessel.

2. The device of claim 1, further comprising a connecting part connecting the fixed part and the extension part to each other.

3. The device of claim 1, wherein the number of fixed part is one or two or more.

4. The device of claim 1, wherein the fixed part has a ring shape.

5. The device of claim 4, wherein the fixed part has a shape in which a top dead center and a bottom dead center are continuously formed.

6. The device of claim 5, wherein the extension part is connected to the top dead center, the bottom dead center, or any one point of a line connecting the top dead center and the bottom dead center to each other.

7. The device of claim 1, wherein the extension part is formed by an open-type wire.

8. The device of claim 7, wherein the extension part has a straight line shape, a spiral shape, or a curved line shape.

9. The device of claim 1, further comprising a spherical cap or a bent part at one distal end of the extension part.

10. The device of claim 1, wherein the number of extension part is one or two or more.

11. The device of claim 10, wherein the extension part includes a first extension part extending by a predetermined length from the fixed part toward an inflow side of the vessel, and a second extension part extending by a predetermined length from the fixed part toward an outflow side of the vessel.

12. The device of claim 11, further comprising a first connecting part connecting any one point of the fixed part and the first extension part to each other, and a second connecting part connecting any one point of the fixed part and the second extension part to each other.

13. The device of claim 11, wherein the number of the first extension part and the number of the second extension part are one or two or more, respectively.

14. The device of claim 13, wherein the first extension part includes a 1-1-th extension part extending by a predetermined length from any one point of the fixed part, and a 1-2-th extension part extending by a predetermined length from any one point of the fixed part.

15. The device of claim 1, wherein the extension part is formed by a closed-type wire.

16. The device of claim 15, wherein the extension part includes a 1-1-th extension part extending by a predetermined length from any one point of the fixed part toward an inflow side of the vessel, a 1-2-th extension part extending by a predetermined length from any one point of the fixed part toward the inflow side of the vessel, wherein the device further comprising a first body part connecting both distal ends of the 1-1-th extension part and the 1-2-th extension part to each other.

17. The device of claim 16, further comprising a first body connecting part connecting one distal end of the first body part and the 1-1-th extension part to each other, and a second body connecting part connecting the other distal end of the first body part and the 1-2-th extension part to each other.

18. The device of claim 1, wherein the number of fixed part is two or more, and any two fixed parts among a plurality of fixed parts are connected by the extension part.

19. The device of claim 18, further comprising a connecting part connected to the fixed part at each of both distal ends of the extension part.

20. The device of claim 18, wherein the fixed part includes a first fixed part, and a second fixed part installed to be spaced apart from the first fixed part while facing the first fixed part.

21. The device of claim 20, comprising a third connecting part extending by a predetermined length from any one point of the first fixed part, and a fourth connecting part extending by a predetermined length from any one point of the second fixed part, wherein one distal end of the third connecting part and one distal end of the fourth connecting part are connected to the extension part extending by the predetermined length.

22. The device of claim 21, further comprising a first coupling part connecting one distal end of the third connecting part and one end of the extension part to each other, and a second coupling part connecting one distal end of the fourth connecting part and the other end of the extension part to each other.

23. The device of claim 22, wherein the first coupling part and the second coupling part have a curved line shape.

24. The device of claim 18, wherein the extension part has a straight line shape, a curved line shape, an “S”-letter shape, or a spiral shape.

25. A method of introducing a device for changing a course of a vessel, comprising:
compressing the device of claim 1;
introducing the compressed device into a vessel of the brain of a target object; and
expanding the introduced device in a length direction of the vessel.

26. A method for changing a course of a vessel using a device for changing a course of a vessel, comprising:
compressing the device of claim 1;
introducing the compressed device into a vessel of a target object of which a course is to be changed; and
expanding the introduced device in a length direction of the vessel.

27. A treatment method of a neurovascular compression syndrome, the treatment method comprising:
compressing the device of claim 1;
introducing the compressed device into a vessel of the brain of a target object; and
expanding the introduced device in a length direction of the vessel after the introducing.

28. The treatment method of claim 27, wherein the device is introduced into a central portion or a proximal portion of the vessel compressing a nerve.

29. The treatment method of claim 27, wherein it is to treat the neurovascular compression syndrome compressing a nerve, by changing the course of the vessel compressing the nerve.

30. The treatment method of claim 27, wherein the neurovascular compression syndrome is a trigeminal nerve compression syndrome or a facial nerve compression syndrome.

31. The treatment method of claim 30, wherein the trigeminal nerve compression syndrome or the facial nerve compression syndrome is one or more selected from the group consisting of trigeminal neuralgia, hemifacial spasm, blepharospasm, neurologic pain, and headache.
